(19) United States
(12) Patent Application Publication
(10) Pub. No.: US 2018/0303531 A1
(43) Pub. Date: Oct. 25, 2018

(54) SINGLE-HANDED APPLICATOR

(71) Applicants: BAXTER INTERNATIONAL INC., Deerfield, IL (US); BAXTER HEALTHCARE SA, Glattpark (Opfikon) (CH)

(72) Inventors: Paul Sanders, Greendale, WI (US); Timothy Fulghum, Lakemoor, IL (US)

(21) Appl. No.: 15/956,370

(22) Filed: Apr. 18, 2018

Related U.S. Application Data
(60) Provisional application No. 62/489,158, filed on Apr. 24, 2017.

(51) Int. Cl. A61B 17/88

(52) U.S. Cl. CPC A61B 17/8819; A61B 17/8816; A61B 2017/2923; A61L 2400/04; A61B 17/8822

(57) ABSTRACT

A single-handed applicator system includes an applicator, including a body and a bore defined by the body. The bore has a first end and a second end. The applicator includes a trigger, coupled to the body. The applicator includes a gear system, disposed within the body. The gear system engages with both a portion of a removable stylus and the trigger to actuate the trigger and rotate a gear along at least the portion of the removable stylus. The applicator system includes a delivery tube, configured to engage with the applicator at the first end of the bore. The applicator includes the removable stylus, configured to be received by the applicator at the second end of the bore. The removable stylus is configured to extend through both the bore and at least a portion of the delivery tube.

### PRIORITY CLAIM

This application claims priority to U.S. Provisional Application No. 62/489,158, entitled “SINGLE-HANDED APPLICATOR”, filed Apr. 24, 2017, the entire contents of which are incorporated herein by reference and relied upon.

### BACKGROUND

Certain medical products, such as hemostatic matrix materials, have unique physical properties and delivery requirements. For example, hemostatic material may be flowable and administered via a delivery device. The hemostatic material typically needs to be delivered to a precise location within a patient's body. For at least this reason, the delivery device must be easy to use and control. Once delivered, the hemostatic material may undergo a phase change and harden. Thus, the delivery device must be configured for repeated use without being damaged by any phase-changing products such as hemostatic material.

Typical procedures for delivery of a hemostatic material may include loading a delivery tube with hemostatic material. Specifically, a surgeon may load the delivery tube by filling it up, via a syringe. This is a time intensive process. The delivery tube is positioned, by the surgeon, at a location on the patient's body. The surgeon then inserts a stylus, which is concentric with the delivery tube, into the back-end of the delivery tube. By inserting the stylus into the back-end of the delivery tube, hemostatic material is expelled from the front-end of the delivery tube at the location on the patient's body. This procedure requires two-hand implementation: one hand for holding and positioning the delivery tube and one hand for pushing the stylus. A one-handed procedure would be preferable. Moreover, because the stylus does not translate all the way through the delivery tube, a portion of hemostatic material may remain in the delivery tube, thus wasting hemostatic material. Avoiding needless wasting of hemostatic material would be preferable.

Accordingly, an improved method and system for using a single-handed applicator is needed.

### SUMMARY

In an embodiment an improved method and system for using a single-handed applicator is provided. In an example embodiment, an applicator system includes an applicator, a delivery tube, and a removable stylus. The applicator includes a body and a bore defined by the body having a first end and a second end. The bore is configured to receive the removable stylus at the second end. The applicator further includes a trigger coupled to the body and a gear system disposed within the body. The gear system engages with both a portion of the removable stylus and the trigger. By actuating the trigger, a gear rotates along at least the portion of the removable stylus. The delivery tube is configured to engage with the applicator at the first end of the bore. Upon actuation of the trigger, the removable stylus is configured to extend through both the bore and at least a portion of the delivery tube.

In an embodiment, engagement between the delivery tube and the applicator is a luer lock engagement.

In a related embodiment, the luer lock engagement includes a male luer lock fitting on the delivery tube and a female luer lock fitting on the applicator.

In an embodiment, the removable stylus includes a plurality of teeth disposed along a length of the removable stylus.

In a related embodiment, the plurality of teeth disposed along the length of the removable stylus are disposed on one side of the removable stylus.

In another related embodiment, the removable stylus is configured to engage with the gear system of the applicator, such that the plurality of teeth engage with the gear.

In an embodiment, the gear system is adjustable, such that a gear ratio between the plurality of teeth and the gear is adjustable.

In an embodiment, the applicator further includes a loading compartment, such that a portion of the removable stylus not disposed within the bore at the second end of the bore is stored in the loading compartment.

In a related embodiment, the portion of the removable stylus not disposed within the bore at the second end of the bore is stored in a coiled configuration in the loading compartment.

In an embodiment, at least one of the delivery tube and the removable stylus is flexible.

In an embodiment, the removable stylus is configured to be removed from the applicator at the first end of the bore.

In a related embodiment, the removable stylus is configured to be removed from the applicator while extending through at least a portion of the delivery tube.

In an embodiment, the applicator further includes an indicator that indicates an amount of the removable stylus not yet received by the bore.

In a related embodiment, the indicator includes a window on the body of the applicator.

In a related embodiment, the indicator includes physical markings on the removable stylus.

In another example embodiment, an applicator system includes an applicator, a delivery tube, and a removable stylus. The applicator includes a body and a bore defined by the body having a first end and a second end. The bore is configured to receive the removable stylus at the second end. The applicator further includes a trigger coupled to the body and a gear system disposed within the body. The gear system includes a gear, configured to engage with a portion of the removable stylus, a coil spring, configured to engage with both the trigger and the gear, and a release button, configured to engage with the coil spring. By actuating the trigger, the coil spring is rotated to a charged state. By depressing the release button, the coil spring is rotated to an uncharged state, such that the coil spring delivers a rotational force that is transmitted to the gear that rotates along at least the portion of the removable stylus. The delivery tube is configured to engage with the applicator at the first end of the bore. Upon actuation of the trigger, the removable stylus is configured to extend through both the bore and at least a portion of the delivery tube.

In an embodiment, responsive to the release button no longer being depressed, the coil spring is unable to rotate to an uncharged state.

In an embodiment, engagement between the delivery tube and the applicator is a luer lock engagement.

In a related embodiment, the luer lock engagement includes a male luer lock fitting on the delivery tube and a female luer lock fitting on the applicator.

In another example embodiment, a method of applying a material, via a single-handed applicator, includes filling a delivery tube with a viscous material. The method further includes attaching the delivery tube to a first end of an applicator, by a luer lock engagement. The method includes inserting a first end of a removable stylus into a second end of the applicator. The removable stylus includes a plurality of teeth disposed along a length of the removable stylus. The method includes actuating a trigger, such that a gear system disposed within the applicator rotates a gear engaging with the plurality of teeth. By actuating the trigger repeatedly, the removable stylus passes through the applicator, from the second end of the applicator towards the first end of the applicator and through at least a portion of the delivery tube, such that the viscous material is pushed out of the delivery tube by the removable stylus.

It is accordingly an advantage of the present disclosure to improve both precision and control of delivering medical products with a single-handed applicator.

It is another advantage of the present disclosure to improve preparation time associated with readying the delivery device for use.

It is a further advantage of the present disclosure to prevent device damage associated with any phase-changing medical products.

It is yet a further advantage of the present disclosure to reduce wasted material that is associated with incomplete delivery of medical products.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1 is a schematic view of an applicator system, according to an example embodiment of the present disclosure.

FIGS. 2A to 2C are side elevation views of stylus configurations, according to example embodiments of the present disclosure.

FIG. 3 is a flow chart of an example method for applying material with an applicator system, according to an example embodiment of the present disclosure.

FIGS. 4A and 4B illustrate partially exploded side elevation views of an applicator system, according to an example embodiment of the present disclosure.

FIGS. 5A to 5D illustrate partially exploded side elevation views of an applicator system engaging a stylus, according to an example embodiment of the present disclosure.

### DETAILED DESCRIPTION OF EXAMPLE EMBODIMENTS

As discussed briefly above, an improved method and system for using a single-handed applicator includes: improving both precision and control of delivering medical products, improving preparation time associated with readying the delivery device for use, preventing device damage associated with any phase-changing medical products, and reducing wasted material that is associated with incomplete delivery of medical products.

In various medical procedures, such as surgery, medical professionals may require advanced techniques to reduce or stop bleeding of a patient. One such technique involves use of a hemostatic material, such as a flowable hemostatic material. By delivering a specific quantity of a hemostatic material to a site, bleeding at the site can be significantly reduced. Often it is difficult to access the site of bleeding, such as when bleeding is within the patient's body cavity. Thus, an endoscopic applicator is typically used to deliver the hemostatic material to the site.

Typical endoscopic applicators do not provide for single handed use. For example, a typical applicator implements a hollow tube, loaded with hemostatic material, and a stylus that is manually inserted into the hollow tube to dispense the hemostatic material. Manually dispensing the hemostatic material out of the delivery tube in this manner is problematic for a number of reasons. Loading the endoscopic applicator is problematic because a user has to manually load the hollow tube by filling it up, such as via a syringe, which is often time intensive. Positioning the endoscopic applicator is problematic because the user has to hold the hollow tube with one hand, positioning the distal end at the delivery site, while simultaneously holding the stylus with the other hand. Delivery of material via the endoscopic applicator is problematic because the user must simultaneously push the stylus into the hollow tube with one hand, while keeping the other hand still to ensure precise delivery. Control of delivery is problematic because it is difficult, if not impossible, for the user to know the quantity of delivered hemostatic material or rate of delivery (e.g., consistent delivery vs. burst or sputtering delivery) when using the endoscopic applicator.

Moreover, a typical applicator in the configuration described above implements a stylus that is dimensioned shorter than that of the hollow tube (e.g., 3″ shorter). In other words, when fully inserted into the hollow tube, the stylus does not translate all the way through the hollow tube. This means that, even when expelled to the full capability of the device, some hemostatic material remains unused in the hollow tube, effectively becoming waste material. Additionally, the unused material may undergo a phase change, such as hardening in the hollow tube. Fully hardened material can effectively ruin the hollow tube by clogging it and making it unusable for subsequent procedures, such that the hollow tube itself becomes waste material as well.

Thus, a single-handed endoscopic delivery device is needed to ensure proper loading, positioning, delivery, and control of the delivery device for medical products, especially hemostatic material. The device will also reduce waste, by avoiding unnecessary waste of material, such has hemostatic material, and unnecessary waste of devices, such as hardened material ruining the delivery device.

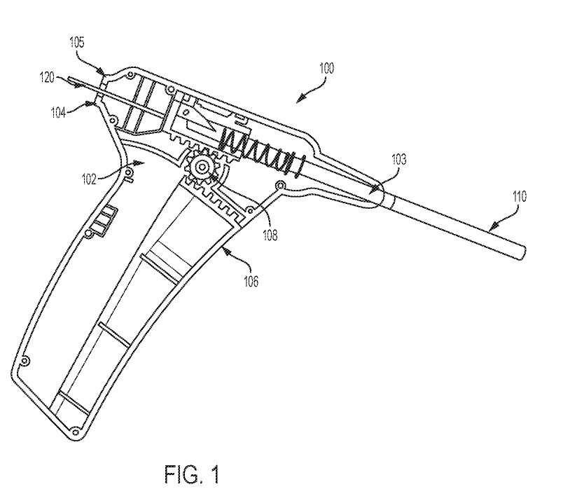

FIG. 1 illustrates a schematic view of an applicator system in accordance with an example embodiment of the present disclosure. The applicator system includes an applicator 100, a delivery tube 110, and a stylus 120. In various embodiments discussed below, it should be appreciated that the stylus 120 is removable from the applicator 100.

More particularly, the applicator 100 includes a body 102, a bore 104, defined by the body 102 having a first end 103 and a second end 105, and a trigger 106, coupled to the body 102. The applicator 100 also includes a gear system 108, disposed within the body 102. The gear system 108 engages with both a portion of the stylus 120 and the trigger 106. By actuating the trigger 106, a drive gear of gear system 108 rotates along at least the portion of the stylus 120. While the disclosure below describes a drive gear rotating along a portion of the stylus 120, it should be appreciated that the drive gear and gear system 108 can include other mechanical components such as additional gears, pawls, and springs; these other mechanical components may, likewise, be used to translate motion from the trigger 106 to the portion of the stylus 120.

In an embodiment, each of the body 102, the trigger 106, the gear system 108, and the stylus 120 are formed via injection molding. In an alternate embodiment, each of the body 102, the trigger 106, the gear system 108, and the stylus 120 are formed via injection molding are formed by 3D printing or other similar plastics manufacturing methods. In an embodiment, each of the body 102, the trigger 106, the gear system 108, and the stylus 120 are manufactured from a polymer. In other embodiments, components may be manufactured from other materials. For example, the gear system 108 may alternatively be manufactured from metal.

The delivery tube 110 is configured to engage with the applicator 100 at the first end 103 of the bore 104. For example, the delivery tube 110 engages with the distal end of the bore 104. In an embodiment, engagement between the delivery tube 110 and the applicator 100 is a luer lock engagement. In a related embodiment, the luer lock engagement includes a male luer lock fitting on the delivery tube 110 and a female luer lock fitting on the applicator 100. In alternate embodiments, engagement between the delivery tube 110 and the applicator 100 may be interference-fit engagement, hook-and-loop engagement, snap engagement, magnetic engagement, or any other sort of mechanical engagement for temporarily affixing the delivery tube 110 to the applicator 100 at the first end 103 of the bore 104.

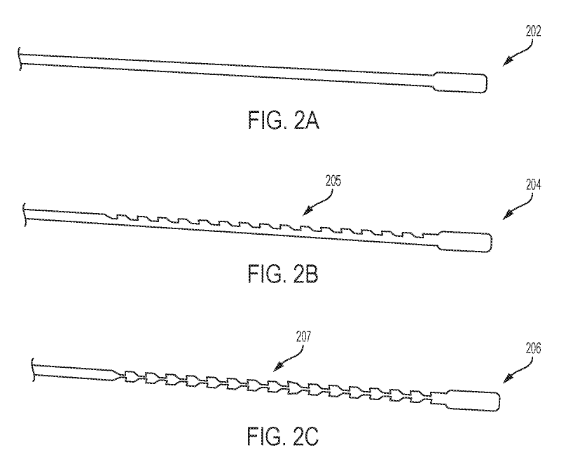

The stylus 120 is configured to be received by the applicator 100 at the second end 105 of the bore 104 and extend through both the bore 104 and at least a portion of the delivery tube 110. For example, the stylus 120 is received at the proximal end of the bore 104. Particular configuration of the stylus 120 is described, in greater detail, with reference to FIGS. 2A to 2C. For example, in an embodiment, the stylus 120 includes a plurality of teeth disposed along a length of the stylus 120. In a related embodiment, the stylus 120 is configured to engage with the gear system 108 of the applicator 100, such that the plurality of teeth engage with the drive gear, the pawl, or any other mechanical components of the gear system 108. By actuating the trigger 106, the drive gear rotates along at least the portion of the stylus 120 of the applicator 100. The stylus 120 engages with this gear such that, responsive to trigger actuation, the stylus 120 advances through the bore 104 from the second end 105 towards the first end 103. For example, a half-squeeze of the trigger 106 advances the stylus 120 by one tooth (e.g., one of the stylus' teeth) through the bore 104; a full-squeeze of the trigger 106 advances the stylus 120 by two teeth (e.g., two of the stylus' teeth) through the bore 104.

The applicator 100 disclosed herein provides for one-handed delivery of hemostatic material. More specifically, single-hand positioning of the applicator 100 and actuation of the applicator 100 via the trigger 106 improves both precision and control of hemostatic material delivery. With applicator 100, the user can position both the applicator 100 and the delivery tube 110 with a single hand, freeing the other hand for other purposes. The user has better control to ensure proper positioning while simultaneously delivering hemostatic material via the trigger 106. Beyond positioning, delivery of hemostatic material is controlled from both a quantity and a rate perspective. Hemostatic material is only delivered when the trigger 106 is pulled; when the trigger 106 is pulled, only a particular amount of hemostatic material is delivered. Consistent trigger actuation will result in a consistent delivery of hemostatic material, thus preventing situations of over-delivery, such as blowout, which can be especially problematic to the patient by causing an embolic event, flushing away previously delivered hemostatic material, or creating other undesirable side effects.

Delivery of hemostatic material via applicator 100 also allows for mechanical optimization of the device itself. For example, applicator 100, by implementing the gear system 108, can generate a greater force than previous delivery techniques such as manual delivery via pushing a stylus through a delivery tube. With a greater delivery force available, applicator dimensions such as the diameter of stylus 120 and/or delivery tube 110 may be reduced. For example, the delivery tube 110 may be longer and narrower when implemented with applicator 100. Reducing particular dimensions of the device, such as the diameter of the delivery tube 110, is a desirable whenever the device is to be used in endoscopic settings. For example, portions of the device, like the delivery tube 110, may pass through an endoscopic port on the patient.

In an embodiment, the gear system 108 of the applicator 100 is adjustable, such that a gear ratio between the plurality of teeth of the stylus 120 and the drive gear is adjustable. For example, as previously noted, the stylus 120 and specifically the plurality of teeth of the stylus 120, engage with the gear system 108 such that, responsive to trigger actuation, the stylus 120 advances through the bore 104 from the second end 105 to the first end 103. In one example, a full-squeeze of the trigger 106 advances the stylus 120 by two teeth. By adjusting the gear ratio, more and/or less of the stylus 120 may translate responsive to trigger actuation. The drive gear and/or a plurality of gears, pawls, and any other mechanical components can be modified so that a new gear ratio is achieved (e.g., between the trigger 106 and the plurality of teeth of the stylus 120). The new gear ratio would mean, for example, that a full-squeeze of the trigger 106 advances the stylus 120 by four teeth (as opposed to advancing the stylus 120 by two teeth with the prior gear ratio). Adjusting the gear ratio affects stylus 120 actuation and, therefore, the quantity of the hemostatic material delivered with each trigger pull. It should be appreciated that modifying the gear ratio also affects the torque being applied to push the material out. For example, the new gear ratio results in a full-squeeze of the trigger 106 advancing the stylus 120 by four teeth; however, the force required to advance the stylus 120 by four teeth might be too high for some users. In other words, there is a delicate balance to be met between the configuration of the gear ratio to advance the stylus 120 to expel material and the ability of the user generate sufficient force to pull the trigger 106.

In an embodiment, the applicator 100 further includes a loading compartment. For example, the loading compartment may be fixed to the proximal end of the applicator 100, such that a portion of the stylus 120 not disposed within the bore 104 at the second end 105 of the bore 104 is stored in the loading compartment. In a related embodiment, the portion of the stylus 120 not disposed within the bore 104 at the second end of the bore 104 is stored in a coiled configuration in the loading compartment. For example, the loading compartment may include the stylus 120 coiled around a spool near the second end 105 of the bore.

In an embodiment, at least one of the delivery tube 110 and the stylus 120 is flexible. In another embodiment, both of the delivery tube 110 and the stylus 120 are flexible. Flexibility may ensure easier delivery of the hemostatic material at particular sites, such as in endoscopic surgical settings when access is restricted through an endoscopic port on the patient.

In an embodiment, after use the stylus 120 is configured to be removed from the applicator 100 at the first end 103 of the bore 104. For example, the stylus 120 is received by the applicator 100 at the second end 105 of the bore 104 (e.g., the proximal end), extends through the bore 104 via trigger actuation, and is removed at the first end of the bore 104 (e.g., the distal end). By having the stylus 120 extend through the bore 104 completely, entering at the second end 105 and exiting at the first end 103, the applicator 100 is configured to prevent device damage associated with material phase-changes such as hemostatic material transitioning from viscous liquid to solid. More specifically, eliminating stylus 120 removal from the second end 105 (e.g., the proximal end) ensures that any hemostatic material touching the stylus 120 travels only in one direction, away from the applicator 100, and is not incidentally dragged back into the applicator 100 by the stylus 120 (e.g., into the gear system 108 of the applicator 100).

In a related embodiment, the stylus 120 is configured to extend through the entire bore 104 and through the entire delivery tube 110. For example, by extending through the entire delivery tube 110, the applicator 100 ensures that all hemostatic material in the delivery tube 110 has been expelled by the stylus 120. This eliminates wasted material issues associated with incomplete delivery of the product. Furthermore, by ensuring complete delivery, none of the hemostatic material remains in the delivery tube 110. This prevents hemostatic material from undergoing a phase change (e.g., hardening) in the delivery tube 110 and rendering the delivery tube 110 as a wasted device.

In another related embodiment, the stylus 120 is configured to be removed from the applicator while extending through at least a portion of the delivery tube 110. For example, when the delivery tube 110 is removed from the applicator 100 (e.g., via the luer lock detachment), the stylus 120 is, likewise, removed. The delivery tube 110 and the stylus 120 can be transported away from the surgical site together. If necessary, a new delivery tube 110 can be attached onto the applicator 100, a new stylus can be inserted into the applicator 100, and additional hemostatic material can be delivered to the surgical site.

For example, the user may intend to use several delivery tubes worth of hemostatic material in a single procedure. In this example, the user will attach a first delivery tube 110, which is loaded with hemostatic material, to the applicator 100 and subsequently deliver hemostatic material to the surgical site by advancing the stylus 120 through the applicator 100 and the delivery tube 110. Once the stylus 120 has extended through the applicator 100 and at least a portion of the delivery tube 110, the delivery tube 110 is detached, and the delivery tube 110 and stylus 120 are transported away from the surgical site. Then, the user will attach a second delivery tube, which is loaded with hemostatic material, to the applicator 100 and subsequently deliver hemostatic material to the surgical site by advancing a second stylus through the applicator 100 and the second delivery tube. In this way, several delivery tubes worth of hemostatic material may be delivered, via applicator 100, in an efficient manner.

The gear system 108 of the applicator 100 may include additional features for delivery control. In an embodiment, the gear system 108 further includes a coil spring and a release button. The coil spring is configured to engage with both the trigger 106 and the drive gear. The release button is configured to engage with the coil spring. By actuating the trigger 106, the coil spring may be rotated to a charged state. By depressing the release button, the coil spring may be rotated to an uncharged state. When rotating from a charged state to an uncharged state, the coil spring effectively delivers a rotational force that is transmitted to the drive gear, such that the drive gear rotates along at least the portion of the stylus 120. In this way, by controlling rotation of the coil spring to the uncharged state (e.g., via the release button), the user can control when the drive gear rotates along the portion of the stylus 120. Thus, via the drive gear and coil spring, the user can control when the stylus 120 is advanced through the bore 104.

In an embodiment, responsive to the release button no longer being depressed, the coil spring is unable to rotate to an uncharged state.

FIGS. 2A to 2C illustrate side elevation views of stylus configurations in accordance with example embodiments of the present disclosure.

For example, FIG. 2A illustrates a first stylus 202. The first stylus 202 includes a first end and a second end. The first stylus 202 is a traditional stylus. For example, first stylus 202 may be manually inserted through a hollow tube (i.e., without use of applicator 100). Prior to insertion, the hollow tube is filled with a viscous material. Thus, by inserting first stylus 202 into the hollow tube, first stylus 202 pushes the viscous material out of the hollow tube. In an embodiment, at least a portion of the stylus 202 has an outer diameter that is equivalent to, or nearly equivalent to, the inner diameter of the hollow tube to facilitate pushing of viscous material.

By comparison, for example, FIG. 2B illustrates a second stylus 204. The second stylus 204 includes a first end, a second end, and a plurality of teeth 205 disposed along a length of the second stylus 204. In an embodiment, the plurality of teeth 205 are disposed along only one side of second stylus 204. Second stylus 204 is configured to be received by the applicator 100 at the second end 105 of the bore 104. Likewise, second stylus 204 is configured to extend through both the bore 104 of the applicator and at least a portion of the delivery tube 110.

Second stylus 204 is configured to engage with the gear system 108 of applicator 100, such that the plurality of teeth 205 engage with the gear system 108. For example, the first end of the second stylus 204 is inserted into the second end 105 of the bore 104 of the applicator 100. The trigger 106 of the applicator 100 is actuated, such that the gear system 108 within the applicator 100 rotates the drive gear engaging with the plurality of teeth 205 of the second stylus 204. By actuating the trigger 106 repeatedly, the second stylus 204 passes through the applicator 100, from the second end 105 of the bore 104 of the applicator 100 towards the first end 103 of the bore 104 of the applicator 100. The second stylus 204 also passes through at least a portion of the delivery tube 110, such that the second stylus 204 pushes the viscous material out of the delivery tube 110. In an embodiment, at least a portion of the second stylus 204 has an outer diameter that is equivalent to, or nearly equivalent to, the inner diameter of the delivery tube 110 to facilitate pushing of viscous material.

Likewise, for example, FIG. 2C illustrates a third stylus 206. The third stylus 206 includes a first end, a second end, and a plurality of teeth 207 disposed along a length of the third stylus 206. In an embodiment, the plurality of teeth 207 are disposed along the entire third stylus 206. For example, the plurality of teeth 207 of the third stylus 206 are along the entire periphery of a length of the third stylus 206, such that a drive gear engages with the teeth 207 along any side of the third stylus 206, as compared to engagement along only one side of the second stylus 204. This allows for the applicator 100 to engage with the stylus 206 along any edge. For example, the stylus 206 can be inserted into the second end 105 of the bore 104 at any rotational orientation. Further, by having the plurality of teeth 207 of the third stylus 206 along the entire periphery of a length of the third stylus 206, the third stylus 206 effectively has a reduced cross-sectional area, which contributes to enhanced flexibility of the third stylus 206.

Third stylus 206 is configured to be received by the applicator 100 at the second end 105 of the bore 104. Likewise, third stylus 206 is configured to extend through both the bore 104 of the applicator and at least a portion of the delivery tube 110.

Third stylus 206 is configured to engage with the gear system 108 of applicator 100, such that the plurality of teeth 207 engage with the gear system 108. For example, the first end of the third stylus 206 is inserted into the second end 105 of the bore 104 of the applicator 100. The trigger 106 of the applicator 100 is actuated, such that the gear system 108 within the applicator 100 rotates the drive gear engaging with the plurality of teeth 207 of the third stylus 206. By actuating the trigger 106 repeatedly, the third stylus 206 passes through the applicator 100 from the second end 105 of the bore 104 of the applicator 100 towards the first end 103 of the bore 104 of the applicator 100. The third stylus 206 also passes through at least a portion of the delivery tube 110, such that the third stylus 206 pushes the viscous material out of the delivery tube 110. In an embodiment, at least a portion of the third stylus 206 has an outer diameter that is equivalent to, or nearly equivalent to, the inner diameter of the delivery tube 110 to facilitate pushing of viscous material.

In an embodiment, applicator 100 may further include an indicator, which indicates an amount of the stylus 120 that has not yet been received by the bore 104 and, consequently, an amount of the stylus 120 that has been received by the bore 104. For example, the indicator includes a window, on the body 102 of the applicator 100, such that the user may visually identify the stylus 120 as the stylus 120 passes through the bore 104 of the applicator. In an embodiment, stylus 120 has teeth, similar to those on second stylus 204 or third stylus 206. Likewise, in an embodiment, the indicator includes physical markings, color indications, or other similar visual indicators. For example, the stylus 120 includes physical markings that are visible outside the bore 104 and/or inside the bore 104 (e.g., via the window described above). The physical markings may be associated with distance and, thus, volume of material pushed by the stylus 120 out of the delivery tube 110. Likewise, in an embodiment, the device may be configured (e.g., via the gear system 108 of applicator 100) to deliver a particular amount of material with each trigger pull. For example, by knowing the distance traveled by the stylus 120 with each trigger pull, and the cross-sectional area of the delivery tube 110, one can easily calculate the volume delivered by applicator 100 with each trigger pull. In a specific example, a half-squeeze of the trigger 106 delivers 5 mL of hemostatic material, whereas a full-squeeze of the trigger 106 delivers 10 mL of hemostatic material.

As described above, the stylus 120 is configured to extend through the bore 104. In an embodiment, the stylus 120 passes through the entire bore 104 of the applicator 100, entering at the second end 105 and exiting at the first end 103. For example, if the stylus 120 were to be removed in the traditional way (e.g., removal from the second end 105 of the bore 104), any hemostatic material on the stylus 120 would come into contact with other components of the applicator 100, such as the gear system 108 and gear, stylus teeth and the portion of the stylus 120 engaging with the gear, the trigger 106, and other related components. Because hemostatic material is a viscous liquid, contact with these components would affect performance of the applicator, and could damage the gear system 108. Moreover, as previously noted, hemostatic material may solidify after undergoing a phase change from viscous liquid to solid. Having hemostatic material solidify on components of the applicator 100, more particularly the gear system 108, would further affect performance of the applicator and, in some cases, render the applicator 100 prematurely nonfunctional.

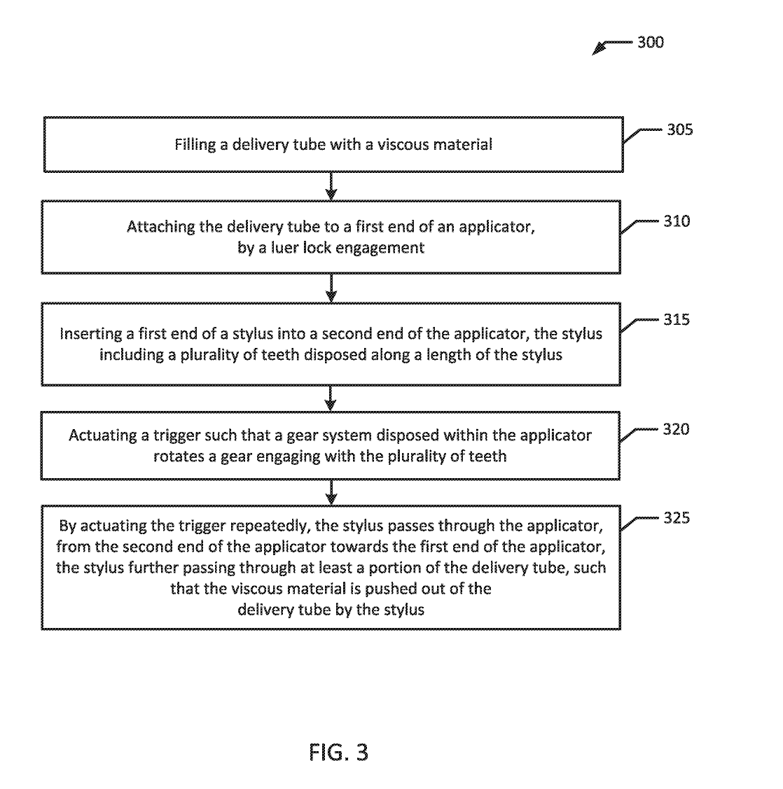

FIG. 3 illustrates a flow chart of an example method for applying material with an applicator system in accordance with an example embodiment of the present disclosure. Although the example method 300 is described with reference to the flowchart illustrated in FIG. 3, it should be appreciated that many other methods of performing the acts associated with the method 300 may be used. For example, the order of some of the blocks may be changed, certain blocks may be combined with other blocks, and some of the blocks described are optional. The method 300 may be performed by processing logic that may include hardware, including circuitry and/or dedicated logic, software, or a combination of both.

The example method 300 starts by filling a delivery tube 110 with a viscous material (block 305). In various embodiments, the delivery tube 110 is loaded by the user and/or come pre-loaded with hemostatic material. Implementing pre-loaded delivery tubes optimizes preparation time and eliminates tedious preparation steps (e.g., loading the tube by filling it up with hemostatic material via a syringe). Example method 300 includes attaching the delivery tube 110 to a first end of an applicator 100, such as the first end 103 of the bore 104, by a threaded engagement such as a luer lock engagement (block 310). For example, the user inserts a male luer lock fitting of the delivery tube 110 (e.g., the proximal end of the delivery tube 110) into the female luer lock fitting on the applicator 100 (e.g., the first end 103 of the bore 104); the user twists the delivery tube 110 to ensure a fixed connection. It should be appreciated that other suitable connection arrangements can be used, besides luer lock fittings, for attaching the delivery tube 110 to the applicator 100.

Next, example method 300 includes inserting a first end of a stylus 120 into a second end of the applicator 100, such as the second end 105 of the bore 104 (block 315). The stylus 120 may include a plurality of teeth disposed along a length of the stylus 120. For example, in one embodiment, the user inserts the stylus 120 into the proximal end of the bore 104 of the applicator 100. The user then positions the distal end of the delivery tube 110 at a delivery site (e.g., on a patient's body).

Example method 300 further includes actuating a trigger 106 such that a gear system 108 disposed within the applicator 100 rotates a gear engaging with the plurality of teeth (block 320). By actuating the trigger 106 repeatedly, the stylus 120 passes through the applicator 100, from the second end 105 of the bore 104 of the applicator 100 towards the first end 103 of the bore 104 of the applicator 100. The stylus 120 also passes through at least a portion of the delivery tube 110, such that the viscous material is pushed out of the delivery tube 110 by the stylus 120 (block 325). For example, the stylus 120 moves from the proximal end of the bore 104 of the applicator 100 to the distal end of the bore 104 of the applicator 100; likewise, the stylus 120 moves through at least a portion of the delivery tube 110, while simultaneously pushing hemostatic material in the delivery tube 110 out of the distal end of the delivery tube 110. The stylus 120 passes through the bore 104 completely and passes through at least a portion of the delivery tube 110. In an embodiment, the stylus 120 passes through the entire delivery tube 110.

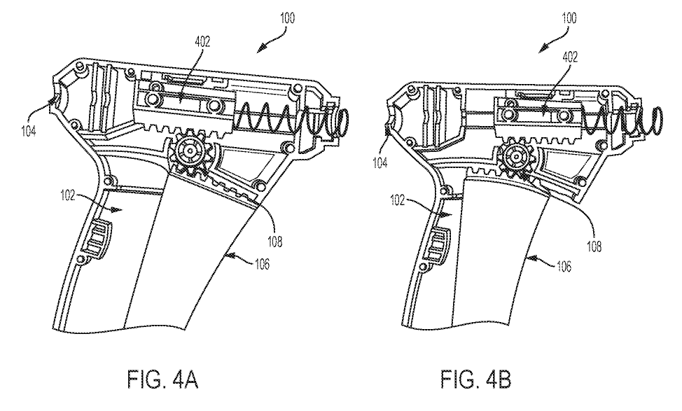

FIGS. 4A to 4B illustrate partially exploded side elevation views of an applicator system in accordance with an example embodiment of the present disclosure. More particularly, as illustrated in FIG. 4A, the applicator system includes the applicator 100, which has the body 102, the bore 104, defined by the body 102 having the first end and the second end, and the trigger 106, coupled to the body 102. The applicator 100 also includes the gear system 108, disposed within the body 102. The gear system 108 engages with both a portion of a stylus (not shown) and the trigger 106.

FIG. 4B illustrates the applicator system responsive to trigger engagement. By actuating the trigger 106, a drive gear of gear system 108 rotates, engaging a pawl 402 that is in contact with at least the portion of the stylus. For example, the gear system 108 translates a directional force, received by the trigger 106, into a rotational force, by a drive gear, and subsequently back into a directional force on the stylus, by the pawl 402, which may be a spring-loaded pawl. For example, in an embodiment, the pawl 402 may include a mechanical dog that engages with the stylus (e.g., with the teeth of the stylus) when the pawl 402 translates in a distal direction. Likewise, the pawl 402 may be spring-loaded, such that the pawl 402 is biased in a particular direction. For example, by biasing the pawl 402 in a particular direction, the gear system 108 and trigger 106 may, likewise, be biased in a particular direction. Further, the pawl 402 may engage with the body 102, such as via grooves on the inside of the body 102, such that the pawl 402 only translates in one linear direction (e.g., proximally and distally in the direction of the stylus 120). In this embodiment, the pawl 402 and mechanical dog may not engage with the stylus when the pawl 402 translates in a proximal direction. In this way, actuation of the stylus is limited to one direction: a distal direction. In a different embodiment, the gear system 108 engages directly with the stylus (e.g., with the teeth of the stylus) to facilitate translation of the stylus.

While the gear system 108 depicted in FIGS. 4A to 4B includes the drive gear and the pawl 402, it should be appreciated that the gear system 108 can include other mechanical components such as additional gears, pawls, and springs; these other mechanical components may be used to translate motion from the trigger 106 to the portion of the stylus. Thus, regardless of the particular configuration, as the trigger 106 is actuated, the gear system 108 will translate motion to the stylus.

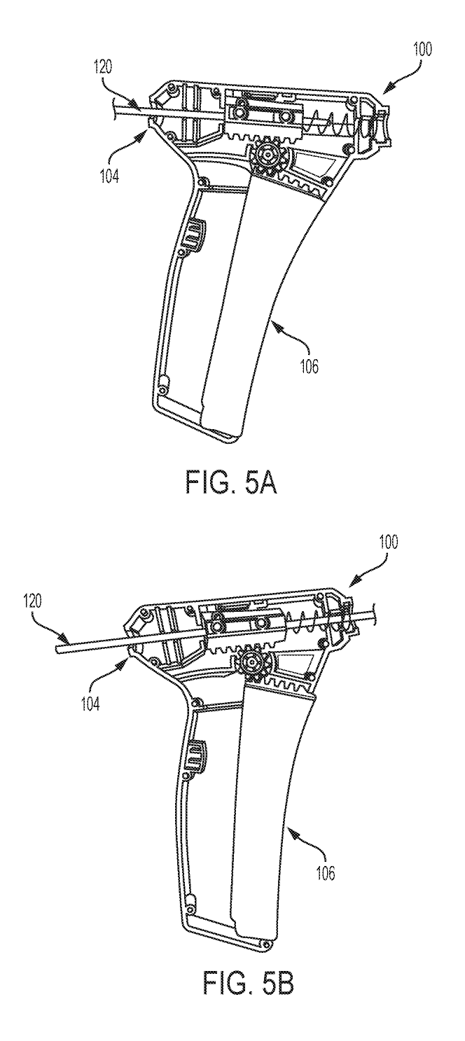

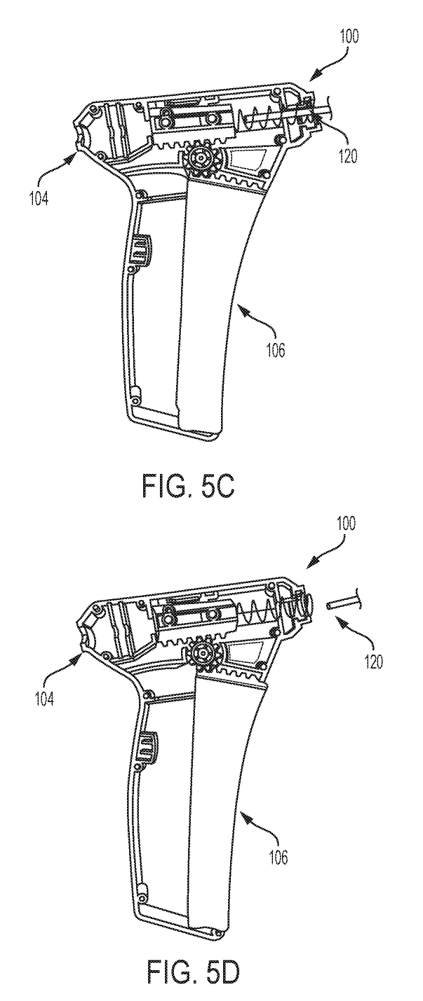

FIGS. 5A to 5D illustrate partially exploded side elevation views of an applicator system engaging a stylus, according to an example embodiment of the present disclosure. For example, in FIG. 5A, a delivery tube 110 is configured to engage with the applicator 100 at the first end 103 of the bore 104 (as previously described above). As illustrated in FIG. 5A, the stylus 120 is configured to be received by the applicator 100 at the second end 105 of the bore 104.

As illustrated in FIG. 5B, the stylus 120 is configured to extend through both the bore 104 and at least a portion of the delivery tube 110 (not shown). More particularly, by actuating the trigger 106, the drive gear rotates, engaging the pawl 402 that is disposed along at least the portion of the stylus 120 of the applicator 100. For example, the pawl 402 may be in contact with a portion of the stylus 120. The stylus 120 engages with this pawl 402 such that, responsive to trigger actuation, the stylus 120 advances through the bore 104 from the second end 105 toward the first end 103. FIG. 5C, likewise, shows a third configuration where the stylus 120 has advanced even further through both the bore 104 and at least a portion of the delivery tube.

FIG. 5D illustrates a fourth configuration where the stylus 120 has advanced all the way through the bore 104. In this fourth configuration, the stylus 120 is entirely disposed within the delivery tube 110, such that none of the stylus 120 remains within the bore 104. For example, the stylus 120 may be removed from the applicator 100 at the first end of the bore 104. In this way, the applicator 100 is configured to prevent device damage associated with material phase-changes, such as hemostatic material transitioning from viscous liquid to solid. By eliminating stylus 120 removal at the proximal end of the applicator 100, the applicator 100 ensures that the hemostatic material travels only in one direction, a distal direction, and is not incidentally dragged back into the applicator 100 and related components such as the gear system 108 and the spring loaded pawl 402.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

The invention is claimed as follows:

1. An applicator system comprising:
an applicator, including:
a body,
a bore defined by the body, wherein the bore has a first end and a second end and is configured to receive a removable stylus at the second end,
a trigger coupled to the body, and
a gear system disposed within the body, wherein the gear system engages with both a portion of the removable stylus and the trigger such that, by actuating the trigger, a gear rotates along at least the portion of the removable stylus; and
a delivery tube, configured to engage with the applicator at the first end of the bore,
wherein, upon actuation of the trigger, the removable stylus is configured to extend through both the bore and at least a portion of the delivery tube.

2. The applicator system of claim 1, wherein engagement between the delivery tube and the applicator is a luer lock engagement.

3. The applicator system of claim 2, wherein the luer lock engagement includes a male luer lock fitting on the delivery tube and a female luer lock fitting on the applicator.

4. The applicator system of claim 1, wherein the removable stylus includes a plurality of teeth disposed along a length of the removable stylus.

5. The applicator system of claim 4, wherein the plurality of teeth disposed along the length of the removable stylus are disposed on one side of the removable stylus.

6. The applicator system of claim 4, wherein the removable stylus is configured to engage with the gear system of the applicator, such that the plurality of teeth engage with the gear.

7. The applicator system of claim 4, wherein the gear system is adjustable, such that a gear ratio between the plurality of teeth and the gear is adjustable.

8. The applicator system of claim 1, wherein the applicator further includes a loading compartment, such that a portion of the removable stylus not disposed within the bore at the second end of the bore is stored in the loading compartment.

9. The applicator system of claim 8, wherein the portion of the removable stylus not disposed within the bore at the second end of the bore is stored in a coiled configuration in the loading compartment.

10. The applicator system of claim 1, wherein at least one of the delivery tube and the removable stylus is flexible.

11. The applicator system of claim 1, wherein the removable stylus is configured to be removed from the applicator at the first end of the bore.

12. The applicator system of claim 11, wherein the removable stylus is configured to be removed from the applicator while extending through at least a portion of the delivery tube.

13. The applicator system of claim 1, further comprising an indicator that indicates an amount of the removable stylus not yet received by the bore.

14. The applicator system of claim 13, wherein the indicator includes a window on the body of the applicator.

15. The applicator system of claim 13, wherein the indicator includes physical markings on the removable stylus.

16. An applicator system comprising:
an applicator, including:
a body,
a bore defined by the body, wherein the bore has a first end and a second end and is configured to receive a removable stylus at the second end,
a trigger coupled to the body, and
a gear system disposed within the body, wherein the gear system includes:
a gear, configured to engage with a portion of the removable stylus,
a coil spring, configured to engage with both the trigger and the gear, and
a release button, configured to engage with the coil spring,
wherein, by actuating the trigger, the coil spring is rotated to a charged state, and
wherein, by depressing the release button, the coil spring is rotated to an uncharged state, such that the coil spring delivers a rotational force that is transmitted to the gear, such that the gear rotates along at least the portion of the removable stylus; and
a delivery tube, configured to engage with the applicator at the first end of the bore,
wherein upon actuation of the trigger, the removable stylus is configured to extend through both the bore and at least a portion of the delivery tube.

17. The applicator system of claim 16, wherein responsive to the release button no longer being depressed, the coil spring is unable to rotate to an uncharged state.

18. The applicator system of claim 16, wherein engagement between the delivery tube and the applicator is a luer lock engagement.

19. The applicator system of claim 18, wherein the luer lock engagement includes a male luer lock fitting on the delivery tube and a female luer lock fitting on the applicator.

20. A method of applying a material comprising:
filling a delivery tube with a viscous material;
attaching the delivery tube to a first end of an applicator, by a luer lock engagement;
inserting a first end of a removable stylus into a second end of the applicator, the removable stylus including a plurality of teeth disposed along a length of the removable stylus; and
actuating a trigger such that a gear system disposed within the applicator rotates a gear engaging with the plurality of teeth,
wherein, by actuating the trigger repeatedly, the removable stylus passes through the applicator, from the second end of the applicator towards the first end of the applicator, the removable stylus further passing through at least a portion of the delivery tube, such that the viscous material is pushed out of the delivery tube by the removable stylus.
